(19) United States
(12) Patent Application Publication
(10) Pub. No.: US 2008/0232131 A1
(43) Pub. Date: Sep. 25, 2008

(54) LIGHT SOURCE DEVICE OF ENDOSCOPE SYSTEM

(75) Inventor: Tadaaki Suda, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 12/052,029

(22) Filed: Mar. 20, 2008

(30) Foreign Application Priority Data
Mar. 23, 2007 (JP) 2007-075930

(51) Int. Cl. A61B 1/06

(52) U.S. Cl. 362/574

(57) ABSTRACT

A light source device of an endoscope system comprises a light-source unit, a light-sensitive element, and a driving unit. The light-source unit emits light and supplies light to the photographic subject through an electronic scope. The light-sensitive element receives light emitted from the light-source unit. The driving unit adjusts the driving intensity of the light-source unit on the basis of a first information regarding an emitting-light intensity of the light-source unit output from the light-sensitive element.

### BACKGROUND OF THE INVENTION

1. Field of the Invention

The present invention relates to a light source device of an endoscope system that adjusts the emitting-light intensity of the light source.

2. Description of the Related Art

An endoscope system that has an electronic scope including an imaging sensor is proposed.

Japanese unexamined patent publication (KOKAI) No. 2006-006832 discloses an endoscope system that adjusts the emitting-light intensity of a light source device including a light-source unit such as a lamp and mechanical parts such as an aperture. In that endoscope system, the emitting-light intensity of the light source device is adjusted by the aperture without changing the emitting-light intensity of the lamp.

Since mechanical parts are used for adjusting the emitting-light intensity of the light-source device, the light-source device must be large.

### SUMMARY OF THE INVENTION

Therefore, an object of the present invention is to provide a light source device for an endoscope system that adjusts the emitting-light intensity of the light source device, without enlarging the light source device.

According to the present invention, a light source device of an endoscope system comprises a light-source unit, a light-sensitive element, and a driving unit. The light-source unit emits light and supplies light to the photographic subject through an electronic scope. The light-sensitive element receives light emitted from the light-source unit. The driving unit adjusts the driving intensity of the light-source unit on the basis of a first information regarding an emitting-light intensity of the light-source unit output from the light-sensitive element.

### BRIEF DESCRIPTION OF THE DRAWINGS

The objects and advantages of the present invention will be better understood from the following description, with reference to the accompanying drawings in which:

FIG. 1 is a construction diagram of the endoscope system in the first embodiment;

FIG. 2 is a construction diagram of the LED driver in the first embodiment;

FIG. 3 is a construction diagram of the endoscope system in the second embodiment;

FIG. 4 is a construction diagram of the LED driver in the second embodiment; and

FIG. 5 is a distribution map of the amplification weighting on the display area of the monitor.

### DESCRIPTION OF THE PREFERRED EMBODIMENTS

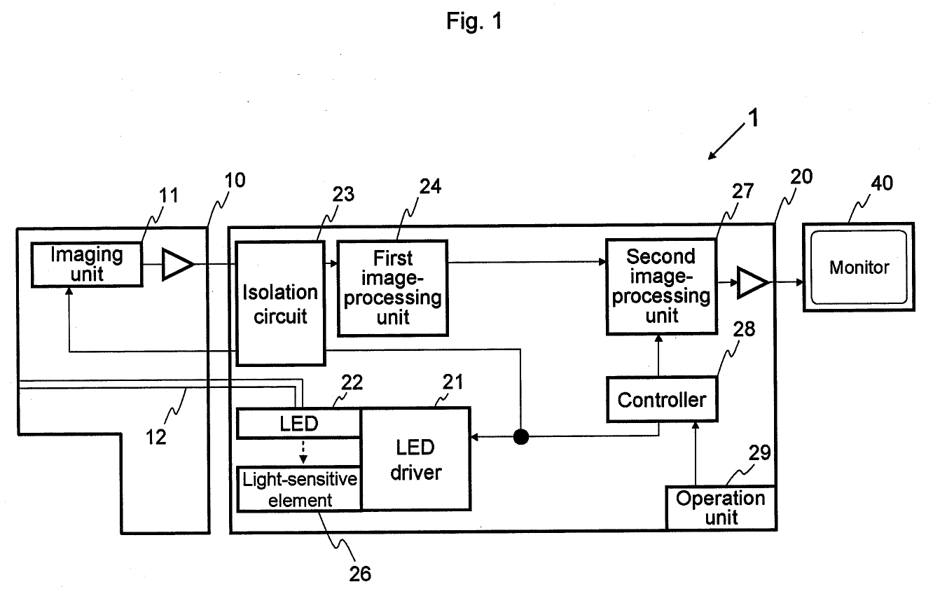

The present invention is described below with reference to the embodiments shown in the drawings. As shown in FIG. 1, an endoscope system 1 in the first embodiment comprises an electronic scope 10, an image processor 20, and a monitor 40.

The electronic scope 10 has an insertion part and an operation-connection part. The insertion part is a flexible tube and is inserted into the body of a patient. The tip of the insertion part has an imaging unit 11 that has an imaging sensor and a control circuit for the imaging sensor. The operation-connection part has an operation key and is connected to the image processor 20.

During operation, the operator of the electronic scope 10 holds the operation-connection part and operates the operation key of the operation-connection part.

The electronic scope 10 has a light guide 12 that guides light from the image processor 20 to the tip of the insertion part through the operation-connection part.

The image processor 20 has an LED driver 21, an LED 22, an isolation circuit 23, a first image-processing unit 24, a light-sensitive element 26, a second image-processing unit 27, a controller 28, and an operation unit 29. The image processor 20 performs image-processing on the image signal obtained by the electronic scope 10 so that the image corresponding to the image signal can be displayed on the monitor 40.

The monitor 40 is connected to the image processor 20. The monitor 40 displays the image in conformity with the standard of the predetermined video signal, upon which the image-processing is performed by the image processor 20.

The external memory that stores the image data, etc., based on the image signal upon which image-processing is performed by the image processor 20, may be connected to the image processor 20. Furthermore, the printer, that outputs the image based on the image signal upon which image-processing is performed by the image processor 20, may be connected to the image processor 20.

Next, the details of the endoscope system 1 are explained.

The light emitted by the LED 22 is cast on the photographic subject through the light guide 12 which is provided in the electronic scope 10 and has many optical fibers. Furthermore, the light emitted by the LED 22 is cast on the light-sensitive element 26 that is arranged near the LED 22.

In the first embodiment, the image processor 20 includes a light-source unit such as the LED driver 21 and the LED 22, etc. However, the light-source unit may be separate from the image processor 20.

Furthermore, the light source of the light-source unit is not limited to the LED, such as in the case in which the emitting-light intensity of the light source can be adjusted by adjusting the driving intensity of the light source.

The LED 22 is driven by the LED driver 21 which is controlled by the controller 28.

The driving intensity of the LED 22 is adjusted by the light-sensitive element 26 that detects the emitting-light intensity of the LED 22 and the LED driver 21.

Representing the driving intensity of the LED 22, the value of the current that passes through the LED 22 is adjusted by the light-sensitive element 26 and the LED driver 21, when the LED 22 is driven by continuous passage of electric current to the LED 22, in other words, by a current drive.

Representing the driving intensity of the LED 22, the value of the duty ratio of the pulse is adjusted by the light-sensitive element 26 and the LED driver 21, when the LED 22 is driven in a pulse drive based on the supply of a pulse train.

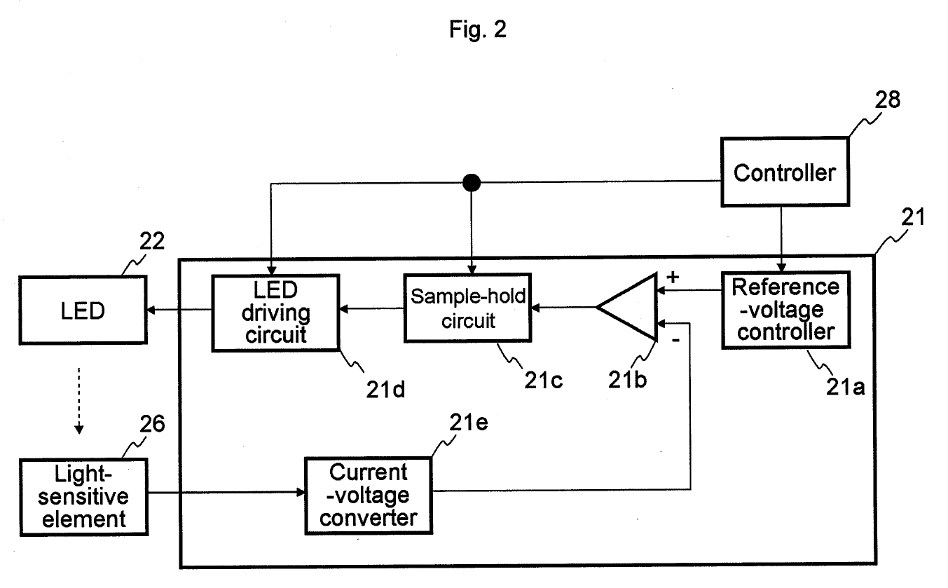

The LED driver 21 has a reference-voltage controller 21a, a comparator 21b, a sample-hold circuit 21c, an LED driving circuit 21d, and a current-voltage converter (an I/V converter) 21e (see FIG. 2).

The reference-voltage controller 21a outputs a first reference voltage to the positive terminal of the comparator 21b, in other words, the first reference voltage is applied to the positive terminal of the comparator 21b, by the reference-voltage controller 21a.

The first reference voltage corresponds to the set value of the emitting-light intensity of the LED 22 set by the operator using the operation unit 29.

The first reference voltage is calculated based on the relationship between the set value of the emitting-light intensity of the LED 22, the current value output from the light-sensitive element 26, and the voltage value output from the current-voltage converter 21e.

The control signal corresponding to the set value of the emitting-light intensity of the LED 22 set by the operator using the operation unit 29, is output from the controller 28 to the reference-voltage controller 21a, so that the reference-voltage controller 21a outputs the first reference voltage corresponding to the control signal.

The light-sensitive element 26 receives the light emitted from the LED 22 and outputs to the current-voltage converter 21e, a current commensurate with the received-light intensity that is received by the light-sensitive element 26.

The current-voltage converter 21e converts the current (the received-light current) output from the light-sensitive element 26 to a voltage (the received-light voltage) and outputs the received-light voltage to the negative terminal of the comparator 21b; in other words, the received-light voltage is applied to the negative terminal of the comparator 21b by the current-voltage converter 21e.

The comparator 21b compares the value of the first reference voltage that is applied to the positive terminal with the value of the received-light voltage that is applied to the negative terminal, and outputs a binary data signal to the sample-hold circuit 21c.

Specifically, when the value of the received-light voltage is lower than the value of the first reference voltage, a low signal is output as a binary data signal.

When the value of the received-light voltage is higher than the value of the first reference voltage, a high signal is output as a binary data signal.

The sample-hold circuit 21c increases the voltage of the analog signal that is output from the sample-hold circuit 21c to the LED driving circuit 21d when the binary data signal output from the comparator 21b is the low signal.

The sample-hold circuit 21c decreases the voltage of the analog signal that is output from the sample-hold circuit 21c to the LED driving circuit 21d when the binary data signal output from the comparator 21b is the high signal.

The LED driving circuit 21d supplies the current corresponding to the analog signal from the sample-hold circuit 21c to the LED 22.

When driving of the LED 22 by current commences, the emitting-light intensity of the LED 22 is gradually increased up to the emitting-light intensity corresponding to the first reference voltage and then the emitting-light intensity of the LED 22 is maintained.

Furthermore, because the value of the current that the LED driving circuit 21d supplies to the LED 22 is adjusted according to the received-light intensity at the light-sensitive element 26, the LED 22 emits with constant emitting-light intensity corresponding to the first reference voltage, even if the emitting-light intensity of the LED 22 has deteriorated with age.

The emitting-light intensity of the LED 22 can also be calculated on the basis of the brightness of the image obtained by the imaging unit 11 in the primary image processing by the first image-processing unit 24, the secondary image processing by the second image-processing unit 27, or the video signal processing operation by the second image-processing unit 27, without using the light-sensitive element 26.

However, it may not be known whether the change of the brightness of the image is due to the emitting-light intensity of the LED 22 or some other reason. Another possible reason could be a change in photographic subject or in the performance of the imaging unit 11, etc. Therefore, the emitting-light intensity of the LED 22 can not be stably adjusted solely on the basis of the brightness of the image.

In the first embodiment, the emitting-light intensity of the LED 22 is adjusted so that the emitting-light intensity is constant.

Furthermore, the period for accumulating the electrical charge of the imaging sensor is adjusted by the electrical shutter of the imaging sensor in order to maintain the brightness of the image which is displayed on the monitor 40, corresponding to the change of the brightness of the image caused by a reason other than a change in the emitting-light intensity of the LED 22.

Furthermore, the devices for the adjustment of the emitting-light intensity of the LED 22 consist of electrical circuits, such as the light-sensitive element 26, etc. Therefore, the construction of devices for the adjustment of the emitting-light intensity of the LED 22 can be simplified compared to that of devices with mechanical parts such as an aperture of the light source, etc.

Furthermore, the comparator 21b may output the binary data signal to the controller 28. In this case, the controller 28 can display a warning on the display 40, etc., when the binary data signal goes low for a predetermined time, in other words, when the first reference voltage is higher than the received-light voltage for a predetermined time length. A possible warning would be “The LED 22 should be replaced because it can not emit at the predetermined emitting-light intensity even when driven by the maximum current from the LED driving circuit 21d”.

Therefore, the operator can continue to use the LED 22 up to the last minute when the LED 22 can no longer emit at the predetermined emitting-light intensity.

Accordingly, the light-source unit can be used more effectively compared to an embodiment in which the life span of the light source such as a lamp, etc., is determined on the basis of hours used.

The reflection of the photographic subject based on the illumination of the endoscope system 1 reaches the imaging sensor of the imaging unit 11 through the objective optical system (not depicted), and the optical image of the subject is imaged on the incident surface of the imaging sensor of the imaging unit 11. At the imaging sensor, the photoelectric conversion operation of the optical image is performed and then the image signal based on the optical image is output.

The image signal output from the imaging unit 11 is amplified and then transmitted to the first image-processing unit 24 of the image processor 20 through the isolation circuit 23. The first image-processing unit 24 performs the primary image processing of the image signal, such as the YC separation that separates the luminance (Y) signal and the chrominance (C) signal of the image signal, etc. The isolation circuit 23 protects the patient from electric shock, etc.

Then the second image-processing unit 27 performs the secondary image processing of the image signal on which the primary image processing is applied, such as amplification, gamma correction, edge enhancement, etc., and then temporarily stores the image data based on the image signal in the memory (not depicted).

The image data temporarily stored in the memory of the second image-processing unit 27 is read in order to perform the video signal processing operation in conformity with the standard of the predetermined video signal and then output to the monitor 40. Thus, an image corresponding to the photographic subject is displayed on the monitor 40.

The controller 28 is a microprocessor or the like, that controls all parts of the electronic scope 10 and the image processor 20.

The operation unit 29 is an input device used for setting the use conditions of the parts of the electronic scope 10 and the image processor 20, etc. Specifically, the operation unit 29 is used for setting the set value of the emitting-light intensity of the LED 22, that corresponds to the first reference voltage. By operating the operation unit 29, the emitting-light intensity of the LED 22 is adjusted.

Next, the second embodiment is explained. In the first embodiment, the emitting-light intensity of the LED 22 is adjusted on the basis of the first reference voltage that corresponds to the set value of the emitting-light intensity of the LED 22 that is set by the operator using the operation unit 29.

However, in the second embodiment, the emitting-light intensity of the LED 22 is adjusted on the basis of a second reference voltage. The second reference voltage is set on the basis of the first reference voltage and the luminance signal included in the video signal that is generated by the video signal processing operation by the second image-processing unit 27, in other words, information regarding the brightness of the image. The points that differ from the first embodiment are explained next.

In the second embodiment, the image processor 20 has the LED driver 21, the LED 22, the isolation circuit 23, the first image-processing unit 24, the light-sensitive element 26, the second image-processing unit 27, the controller 28, and the operation unit 29, similar to the first embodiment.

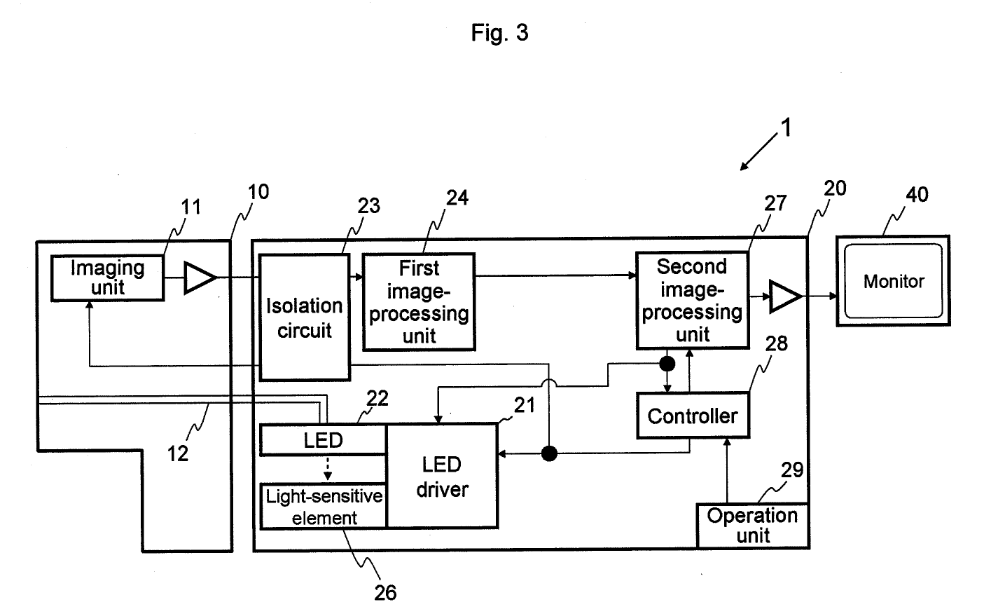

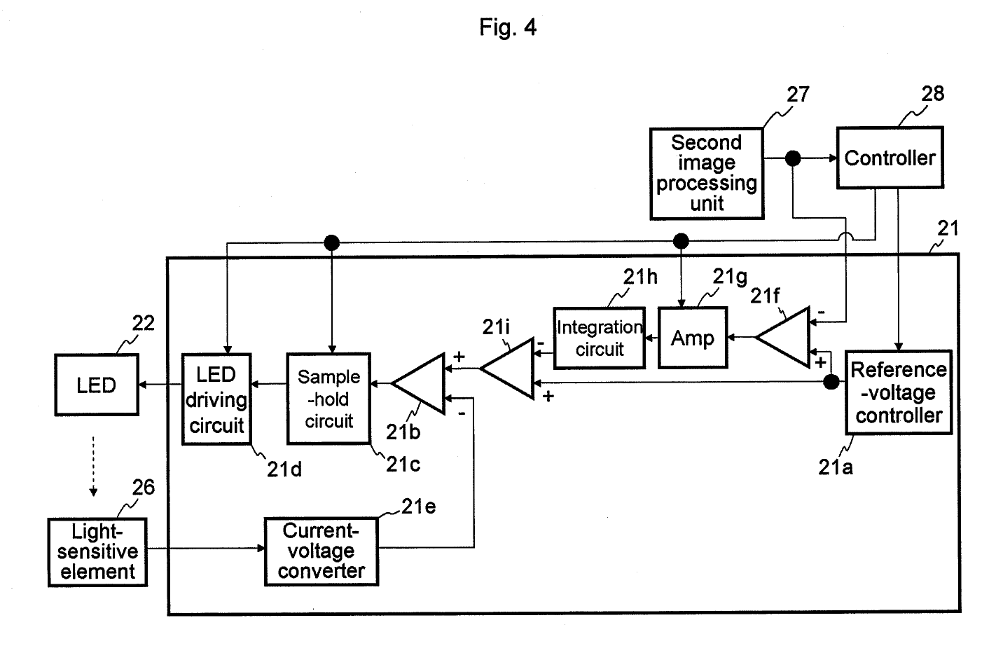

However, in the second embodiment, the luminance voltage corresponding to the luminance signal included in the video signal that is generated by the video signal processing operation by the second image-processing unit 27 is applied to the controller 28 and the positive terminal of the subtraction circuit 21f of the LED driver 21 (see FIGS. 3 and 4).

The LED 22 is driven by the LED driver 21 which is controlled by the controller 28.

The driving intensity of the LED 22 is adjusted by the second image-processing unit 27 that outputs the luminance signal, the light-sensitive element 26 that detects the emitting-light intensity of the LED 22, and the LED driver 21.

Representing the driving intensity of the LED 22, the value of the current that passes through the LED 22 is adjusted by the second image-processing unit 27, the light-sensitive element 26, and the LED driver 21, when the LED 22 is driven by continuous passage of electric current to the LED 22, in other words, by a current drive.

Representing the driving intensity of the LED 22, the value of the duty ratio of the pulse is adjusted by the second image-processing unit 27, the light-sensitive element 26, and the LED driver 21, when the LED 22 is driven in a pulse drive based on the supply of a pulse train.

The LED driver 21 has the reference-voltage controller 21a, the comparator 21b, the sample-hold circuit 21c, the LED driving circuit 21d, and the current-voltage converter (an I/V converter) 21e (see FIG. 4). The LED driver 21 also has a subtraction circuit 21f, a gain-changeable amplifier 21g, an integration circuit 21f, and an adding circuit 21i.

The reference-voltage controller 21a outputs the first reference voltage to the positive terminal of the subtraction circuit 21f and the positive terminal of the adding circuit 21i, in other words, the first reference voltage is applied to the positive terminal of the subtraction circuit 21f and the positive terminal of the adding circuit 21i, by the reference-voltage controller 21a.

The first reference voltage corresponds to the set value of the emitting-light intensity of the LED 22 set by the operator using the operation unit 29.

The first reference voltage is calculated on the basis of the relationship between the set value of the emitting-light intensity of the LED 22, the current value output from the light-sensitive element 26, and the voltage value output from the current-voltage converter 21e.

The control signal corresponding to the set value of the emitting-light intensity of the LED 22 set by the operator using the operation unit 29, is output from the controller 28 to the reference-voltage controller 21a, so that the reference-voltage controller 21a outputs the first reference voltage corresponding to the control signal.

The luminance signal output from the second image-processing unit 27 is input to the negative terminal of the subtraction circuit 21f, in other words, the luminance voltage corresponding to the luminance signal is applied to the negative terminal of the subtraction circuit 21f by the second image-processing unit 27.

The subtraction circuit 21f outputs a differential signal corresponding to the differential voltage between the first reference voltage and the luminance voltage corresponding to the luminance signal to the gain-changeable amplifier 21g.

The gain-changeable amplifier 21g amplifies the differential signal and then outputs it to the integration circuit 21h.

The amplification rate of the differential signal by the gain-changeable amplifier 21g changes all parts of the luminance signal from the imaging area of the imaging sensor of the imaging unit 11, (in other words, all parts of the luminance signal for the display area of the monitor 40,) in order to weight all regions of the image.

Specifically, the amplification rate is set so that the weight of the luminance signal corresponding to the center part of the imaging sensor is enlarged, thus increasing the luminance signal corresponding to the center part 40a of the display area of the monitor 40.

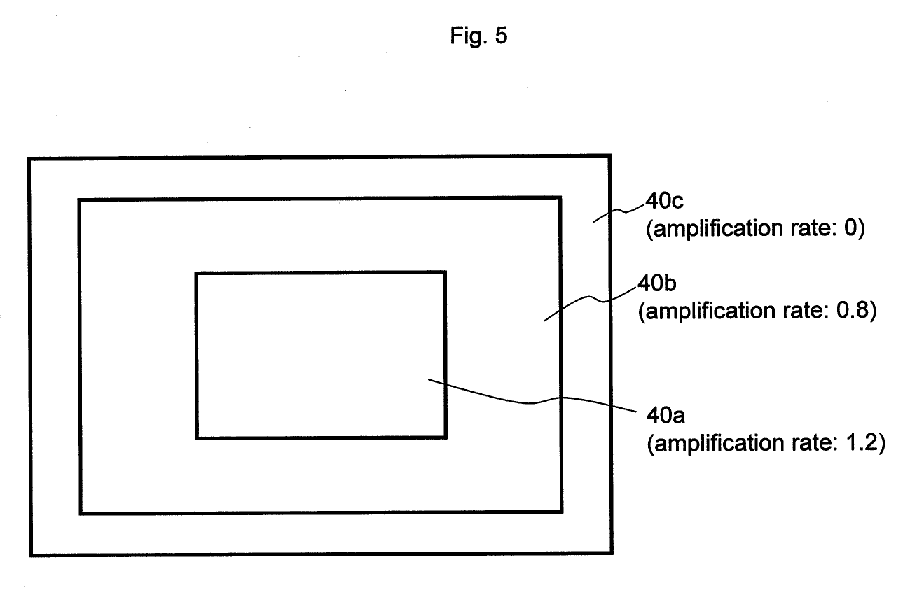

The differential signal based on the luminance signal corresponding to the center part 40a is amplified at a high amplification rate such as 1.2 times the amplification rate (see FIG. 5).

The differential signal based on the luminance signal corresponding to the middle part 40b around the center part 40a is amplified at a middle amplification rate such as 0.8 times the amplification rate.

The differential signal based on the luminance signal corresponding to the circumference part 40c around the middle part 40b is amplified at a low amplification rate such as 0 times the amplification rate.

The interval in the luminance signal corresponding to the display area is specified on the basis of the horizontal line and the horizontal synchronization signal.

Therefore, the information regarding the brightness of the image is used for adjusting the emitting-light intensity of the LED 22, in the case where the brightness of the image at the center of the imaging sensor should be emphasized. Thus, the brightness of the image displayed at the center part 40a of the monitor 40 is emphasized, because the image displayed on the center part 40a is the most important part for observation.

The integration circuit 21h integrates (sums) the differential signal that is amplified at the different amplification rates in all parts of the display area. By integrating, the average voltage of the differential signal is calculated. The integration circuit 21h applies the average voltage on the negative terminal of the adding circuit 21i.

The adding circuit 21i adds the average voltage of the differential signal and the first reference voltage. The second reference voltage is thus calculated. The adding circuit 21i applies the second reference voltage to the positive terminal of the comparator 21b.

The differential voltage is calculated on the basis of the difference between the first reference voltage and the luminance voltage by the subtraction circuit 21f.

The second reference voltage is calculated on the basis of the addition of the amplified differential voltage and the first reference voltage that is used for calculating the differential voltage by the subtraction circuit 21f.

Therefore, when the value of the luminance voltage corresponding to the luminance signal is higher than the value of the first reference voltage, in other words, when the brightness of the actual image is greater than the brightness of the image that is assumed on the basis of the set value of the emitting-light intensity of the LED 22 corresponding to the first reference voltage, a second reference voltage which is lower than the first reference voltage is output from the adding circuit 21i.

When the value of the luminance voltage corresponding to the luminance signal is lower than the value of the first reference voltage, in other words, when the brightness of the actual image is lower than the brightness of the image that is assumed on the basis of the set value of the emitting-light intensity of the LED 22 corresponding to the first reference voltage, a second reference voltage which is higher than the first reference voltage is output from the adding circuit 21i.

The light-sensitive element 26 receives the light emitted from the LED 22 and outputs to the current-voltage converter 21e, a current commensurate with the received-light intensity that is received at the light-sensitive element 26.

The current-voltage converter 21e converts the current (the received-light current) output from the light-sensitive element 26 to a voltage (the received-light voltage) and outputs the received-light voltage to the negative terminal of the comparator 21b; in other words, the received-light voltage is applied to the negative terminal of the comparator 21b by the current-voltage converter 21e.

The comparator 21b compares the value of the second reference voltage that is applied to the positive terminal with the value of the received-light voltage that is applied to the negative terminal, and outputs a binary data signal to the sample-hold circuit 21c.

Specifically, when the value of the received-light voltage is lower than the value of the second reference voltage, a low signal is output as a binary data signal.

When the value of the received-light voltage is higher than the value of the second reference voltage, a high signal is output as a binary data signal.

The sample-hold circuit 21c increases the voltage of the analog signal that is output from the sample-hold circuit 21c to the LED driving circuit 21d when the binary data signal output from the comparator 21b is the low signal.

The sample-hold circuit 21c decreases the voltage of the analog signal that is output from the sample-hold circuit 21c to the LED driving circuit 21d when the binary data signal output from the comparator 21b is the high signal.

The LED driving circuit 21d supplies the current corresponding to the analog signal from the sample-hold circuit 21c to the LED 22.

When the value of the luminance voltage corresponding to the luminance signal is higher than the value of the first reference voltage, in other words, when the brightness of the actual image is higher than the brightness of the image that is assumed on the basis of the set value of the emitting-light intensity of the LED 22 corresponding to the first reference voltage, the adding circuit 21i controls the supply of the current of the LED 22 by the LED driving circuit 21d so that the value of the received-light voltage is close to the value of the second reference voltage which is lower than the value of the first reference voltage.

When the value of the luminance voltage corresponding to the luminance signal is lower than the value of the first reference voltage, in other words, when the brightness of the actual image is lower than the brightness of the image that is assumed on the basis of the set value of the emitting-light intensity of the LED 22 corresponding to the first reference voltage, the adding circuit 21i controls the supply of the current of the LED 22 by the LED driving circuit 21d so that the value of the received-light voltage is close to the value of the second reference voltage which is higher than the value of the first reference voltage.

The other constructions in the second embodiment are the same as those in the first embodiment.

When driving of the LED 22 by current commences, the emitting-light intensity of the LED 22 is gradually increased up to the emitting-light intensity corresponding to the second reference voltage and then the emitting-light intensity of the LED 22 is maintained.

Furthermore, because the value of the current that the LED driving circuit 21d supplies to the LED 22 is adjusted according to the received-light intensity at the light-sensitive element 26, the LED 22 emits with the constant emitting-light intensity corresponding to the second reference voltage, even if the emitting-light intensity of the LED 22 has deteriorated with age.

Furthermore, the adjustment of the emitting-light intensity of the LED 22 also considers the luminance signal included in the video signal, in other words, the brightness of the image displayed on the monitor 40. Therefore, the brightness of the image displayed on the monitor 40 can be maintained at a predetermined level that is close to the brightness of the image that is assumed on the basis of the set value of the emitting-light intensity of the LED 22 corresponding to the first reference voltage, without using the electrical shutter of the imaging sensor.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2007-075930 (filed on Mar. 23, 2007) which is expressly incorporated herein by reference, in its entirety.

1. A light source device of an endoscope system, comprising:
a light-source unit that emits light and supplies light to the photographic subject through an electronic scope;
a light-sensitive element that receives light emitted from said light-source unit; and
a driving unit that adjusts the driving intensity of said light-source unit on the basis of a first information regarding an emitting-light intensity of said light-source unit output from said light-sensitive element.

2. The light source device according to claim 1, wherein said driving unit adjusts said driving intensity of said light-source unit so that the emitting-light intensity of said light-source unit is maintained.

3. The light source device according to claim 1, further comprising an operation unit that is used for setting the set value of the emitting-light intensity of said light-source unit;
wherein said driving unit has a comparing unit and an adjusting unit, said comparing unit comparing a first reference value with said first information, said first reference value corresponding to the set value of the emitting-light intensity of said light-source unit, said adjusting unit adjusting said driving intensity of said light-source unit on the basis of the result of comparison by said comparing unit.

4. The light source device according to claim 3, further comprising:
a controller that controls said driving unit; and
an output device;
wherein said controller displays a warning on said output device, when a difference between said first reference value and said first information persists for a predetermined time length.

5. The light source device according to claim 1, further comprising an operation unit that is used for setting the set value of the emitting-light intensity of said light-source unit;
wherein said driving unit has a comparing unit and an adjusting unit, said comparing unit comparing a second reference value with said first information, said second reference value being set on the basis of a first reference value and a second information regarding the brightness of an image obtained from the reflection of the photographic subject on which the light is cast by said light-source unit, said first reference value corresponding to the set value of the emitting-light intensity of said light-source unit, said adjusting unit adjusting said driving intensity of said light-source unit on the basis of the result of comparison by said comparing unit.

6. The light source device according to claim 5, wherein said second information includes a weight that is applied to all parts of said image.
